(19) United States
(12) Patent Application Publication
(10) Pub. No.: US 2025/0108058 A1
(43) Pub. Date: Apr. 3, 2025

(54) treating breasts tumors and cancer.

(71) Applicant: Menni Menashe Zinger, Ashkelon (IL)

(72) Inventor: Menni Menashe Zinger, Ashkelon (IL)

(21) Appl. No.: 18/375,528

(22) Filed: Oct. 1, 2023

(51) Int. Cl. A61K 31/55

(52) U.S. Cl. CPC A61K 31/55

(57) ABSTRACT

At this invention I present a way to cause tumors to disappear from breast of woman and/or men maybe from other organs too.

At a patient 25 years old at 1999, me, who discovered tumors at breast at two sides after using psychiatric medication that might cause those tumors to appear because of rising of prolactin levels at my body.

after stop taking the psychiatric medication for few mounts and after retaking it together with imipramine/primonil medication at dosage of 50-100 milligrams a day the tumors disappear from breast chest of this man very fast after taking imipramine/primonil and I assumed the taking of imipramine/primonil caused the tumor to disappear and understand the potential of this medication to be a medication for treating breast tumors, cancer and other tumors and cancers at men and woman very usefully.

1. using imipramine/primonil at dosage of 25-200 milligrams a day in order to treat cancer and tumors at breast and other organs at men and or woman by taking pill/s every for a week or few weeks, 2-4 weak in order to keep healthy and preserving treatment in order to keep healthy.
